(19) United States
(12) Patent Application Publication
(10) Pub. No.: US 2006/0173275 A1
(43) Pub. Date: Aug. 3, 2006

(54) System for early detection of disease and development of disease-specific biomarkers

(76) Inventors: Johannes Henricus Joseph Van Nesselrooij, Soest (NL); Robert-Jan Antonius Nicolaas Lamers, Maarssen (NL); Jeroen de Groot, Leiden (NL); Nicole Verzijl, Enschede (NL)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/532,512

(22) PCT Filed: Oct. 27, 2003
(86) PCT No.: PCT/NL03/00728

§ 371 Date: Nov. 30, 2005

(30) Foreign Application Priority Data
Oct. 25, 2002 (NL) 1021753

(51) Int. Cl. A61B 5/05

(52) U.S. Cl. 600/410

(57) ABSTRACT

The present invention relates to a method for the detection of a specific disease in a mammal, comprising providing an NMR spectrum of metabolites in a body fluid of an individual of said mammal in which said disease is suspected and comparing said NMR spectrum with a difference profile, comprising a plurality of NMR spectral line positions, which express the normalized difference between one or more NMR spectra of metabolites in a body fluid of one or more healthy individuals of said mammal, and one or more corresponding NMR spectra of metabolites in a corresponding body fluid of one or more individuals of said mammal in which the disease has been diagnosed. The invention also relates to the said difference profile for the detection of a disease in a mammal and to methods for the manufacture thereof. The invention also relates to a biomarker for detection of specific diseases, in particular osteoarthritis, and to the use of that biomarker in detection of osteoarthritis.

The invention relates to a difference profile between NMR spectra of metabolic metabolites as a pattern for early detection of a disease in a mammal, to a biomarker for detection of a disease, to a method for manufacturing a difference profile and to a method for the identification of biomarkers by means of a difference profile. Finally, the present invention relates to a method for detection of a disease, especially osteoarthritis, in a mammal by means of a biomarker and/or difference profile according to the invention.

In many cases, determining the health condition of an individual can be a difficult matter. Diagnosis is of course difficult if clinical examination reveals no cause of a health problem. Conversely, with some diseases, such as for instance cancer, an individual can have relatively few symptoms, but the disease can nevertheless be at an advanced stage.

For a large number of diseases, such as for instance MS and osteoarthritis, there is not even a diagnostic test (laboratory detection or measurement technique) available at the moment by means of which the disease can be diagnosed with 100% certainty. The diagnosis of such diseases is complex and is often made on the basis of examination of tissue functioning (e.g. nerves and joints respectively) in combination with biochemical and/or pathological tissue examination. This combination is important because, with this type of disorder, clinical examination may show a perfectly normal picture, while the patient already has many symptoms. All the same, the medical specialist will not always be able to diagnose the disease with certainty. Therefore, for an actual diagnosis, it is also necessary to demonstrate progression of the clinical picture in time.

With the existing methods, the progression of the disease cannot be determined or cannot be simply determined. This makes it often impossible to start a therapeutic treatment already at an early stage, so that the disease is often at an advanced stage even before medication is administered.

Also, the absence of early diagnostics limits the development of more specific and more effective therapies. There is a great need for alternative methods which can quantitatively, reliably, sensitively and specifically demonstrate the presence of a disease. Further, there is a need for a method by means of which particular diseases can be diagnosed at an early stage, preferably before the disease process has led to irreversible changes.

The use of molecular markers (or biomarkers) which are specific to the presence of a particular disease could fill this need and can make an important contribution to diagnosis, prognosis and monitoring of the progress of the disease. Further, by means of such molecular markers, research into the effect of clinical treatment therapies and the development of new medicines could be facilitated. Thus, molecular markers are considered crucial for effectively carrying out preclinical studies (both in vitro and in vivo in laboratory animals) and studies directed at the pathophysiology of diseases in general.

An ideal molecular marker is disease-specific, reflects the actual disease activity and/or disease stage, can be used for determining the effectiveness of therapy and contributes to the reliable prognosis of the disease. However, all these requirements do not need to be integrated in one single marker; a combination of complementary markers is possible and could, in particular cases, perform even better.

It is an object of the present invention to provide new systems and methods for the detection of a disease.

Another object of the present invention is to provide systems and methods which solve at least some of the problems associated with existing systems and methods for the detection of a disease as described hereinabove.

Another object of the present invention resides in providing systems and methods as described hereinabove which can be used in in vivo and/or in vitro medical diagnostics.

It has now been found that, in the urine of an individual with a particular disease, such as for instance osteoarthritis, metabolites are present which are not, or in significantly larger or smaller quantities, present in healthy individuals. It was possible to demonstrate the presence of these disease-specific metabolite concentrations by means of a proton nuclear magnetic resonance (¹H NMR) spectroscopic analysis of the metabolites in the urine of mammals. Therefore, these metabolites can be used, individually or in combination, as a biomarker in the diagnostics and prognostics of diseases.

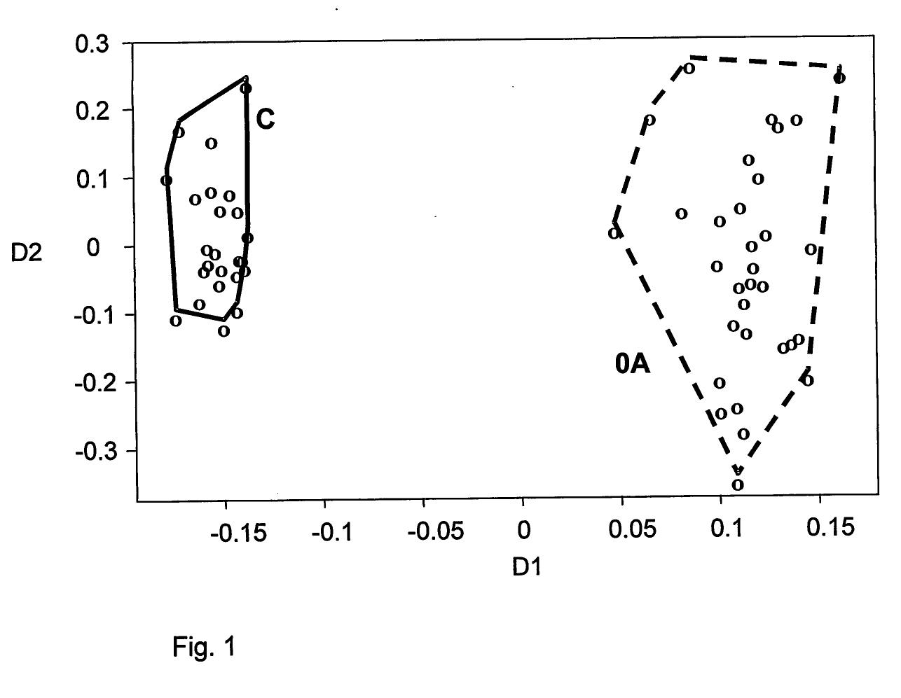

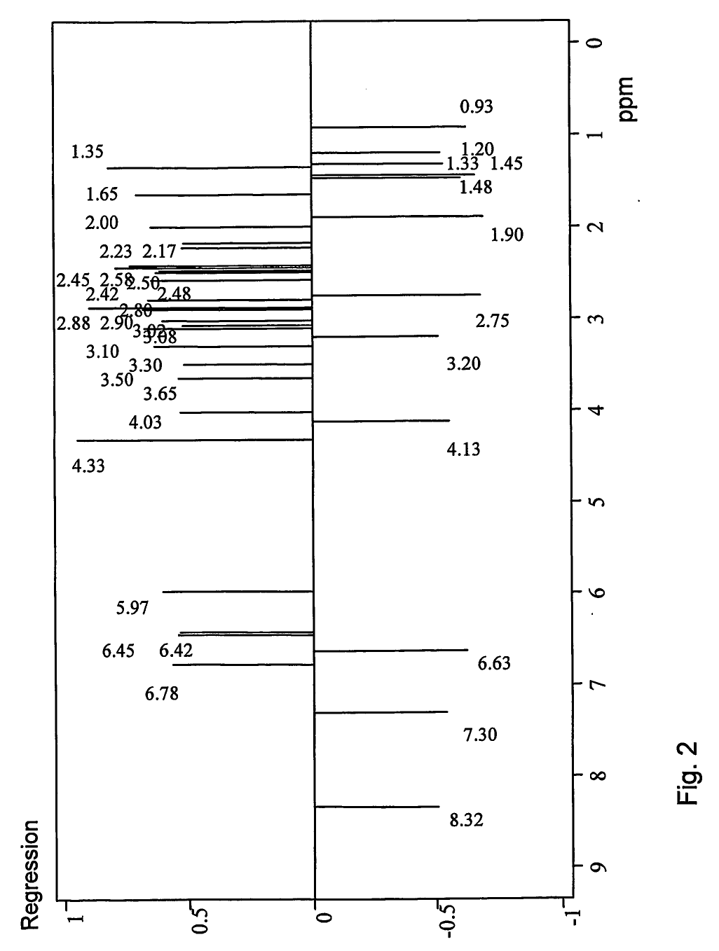

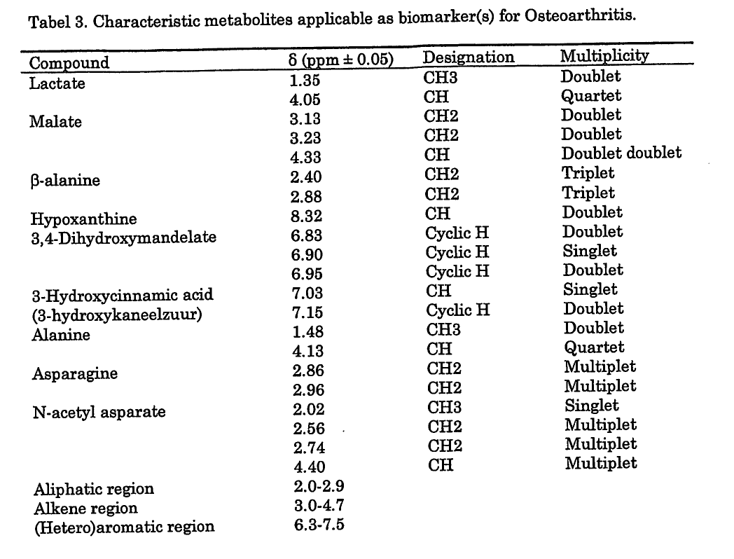

It has further been found that a collection of statistically significant differences between the signal intensity of a large number of spectral lines with defined positions in the NMR spectrum, recorded from metabolites in the urine of a healthy individual, and the signal intensity of corresponding spectral lines in the NMR spectrum recorded from metabolites in the urine of an individual suffering from a specific disorder or disease can provide a pattern which enables the detection of that disease in that individual. In the present invention, this pattern is referred to as a difference profile or metabolic fingerprint. Such a difference profile can be graphically represented as a factor spectrum (see FIGS. 1 and 2).

The present invention therefore relates to a difference profile for the detection of a disease in a mammal, comprising a plurality of spectral line positions and, optionally, corresponding signal intensities of NMR spectral lines, which express the normalized difference between one or more NMR spectra of metabolites in a body fluid of one or more healthy individuals of this mammal, and one or more corresponding NMR spectra of metabolites in a corresponding body fluid of one or more individuals of this mammal in which the disease has already been diagnosed.

FIG. 1 is a representation of a score plot of NMR spectra obtained in the manner as inter alia described in Example 1 for osteoarthritis. On the horizontal axis, component D1 is plotted. On the vertical axis, component D2 is plotted. The left outlined cluster (C) is a cluster of NMR spectra of healthy control individuals, while the right interruptedly outlined cluster (OA) represents a cluster of NMR spectra of patients with osteoarthritis.

FIG. 2 is a representation of a factor spectrum of osteoarthritis obtained in the manner as described in the description below and Example 1. On the horizontal axis, the spectral line position is plotted in “ppm”. On the vertical axis, the signal intensity is plotted in “Regression”.

Osteoarthritis (cartilage degeneration) is one of the most common diseases among elderly people and there is an incidence of more than 50% among people aged 65 years and over. Osteoarthritis is characterized by progressive degradation of articular cartilage and results in impaired movement, pain and ultimately disability. In addition to cartilage degradation, osteoarthritis is pathologically characterized by changes in subchondral bone (sclerosis, cysts), osteophyte formation and mild synovial inflammation. However, the etiology and pathogenesis of osteoarthritis are largely unclear.

The clinical diagnosis of osteoarthritis is based on the observation of clinical symptoms in combination with radiological examination of changes in the joint, especially related to the width of the joint space. However, these changes can only be observed at an advanced stage of the disease. Often, the damage to the joints is already irreversible by then. Since radiological determination of the width of the joint space is relatively insensitive, moreover, after at least 1 year and preferably 2 years, a follow-up examination is needed to determine the progress of the disease and the possible effect of a therapy. This greatly complicates the treatment of osteoarthritis.

Markers which are presently used in osteoarthritis-related examination comprise molecules such as COMP (cartilage oligomeric matrix protein), which, however, is not specific to cartilage, and Glc-Gal-PYD (glucosyl-galactosyl pyridinoline), which is a marker for the degradation of synovial tissue (mucous lining) and can therefore not serve as a diagnostic marker of cartilage degradation. Another marker used is CTX-II, the C-terminal crosslinked telopeptide of type II collagen. This marker is collagen-specific. But since osteoarthritis comprises more than just collagen-related components, use of this marker can yield false negative results.

There is a great need for alternative markers and/or methods which can quantitatively, reliably, sensitively and specifically demonstrate osteoarthritis-related changes in the joints. Further, there is a need for a method by means of which osteoarthritis can be diagnosed at an early stage, preferably before irreversible changes have taken place.

The present application now provides a method for the early detection of a disease in a mammal, such as for instance osteoarthritis, by means of a difference profile between NMR spectra of metabolic metabolites. Such a method is preferably not invasive.

In the present invention, a difference profile is defined as a characteristic selection of NMR spectral lines with defined positions whose values of the signal intensities significantly differ between normalized NMR spectra of metabolites in a body fluid of ill patients and normalized NMR spectra of metabolites in a body fluid of healthy individuals. Such a difference profile comprises the spectral line positions and optionally their corresponding signal intensities or signal intensity differences.

In the present invention, a normalized NMR spectrum is defined as an NMR spectrum in which the diversity or variation in the signal intensities of the spectral lines between samples is limited by arithmetically taking glitches into account. For normalization, the sum of the squares of all intensities is equated with 1. The reason for this is that it is assumed that each sample comprises an equal amount of information. By carrying out normalization, the absolute amount of information in each NMR spectrum is equated (equal surfaces under the NMR spectra), so that they become mutually comparable.

A changing signal intensity of a particular spectral line in two comparable NMR spectra indicates that the concentration of hydrogen atoms in one of those samples has changed and that, thus, the amount of one or more chemical components containing these atoms, in this case metabolites, has changed in one of those samples.

So, a difference profile according to the invention comprises a collection of spectral line positions in a normalized NMR spectrum whose corresponding signal intensity is increased or decreased due to a specific disease compared to the signal intensity in a normalized NMR spectrum of healthy individuals.

Preferably, a difference profile according to the invention comprises spectral line positions whose corresponding signal intensities are increased and/or decreased by a particular factor in the spectrum of an ill patient in relation to a corresponding spectrum of a healthy individual. This factor can be used for applying a (positive) threshold value (or reference value) for increases and a corresponding (negative) threshold value for decreases of the signal intensity. Spectral line positions whose corresponding signal intensities are above or below the corresponding threshold value are included in the difference profile. The endogenous and exogenous metabolites (see below) whose signal intensity cannot be correlated to a healthy or to an ill situation have been eliminated from such a difference profile so that the data are reduced to specific and “significant” disease-related changes.

For eliminating endogenous and exogenous metabolites from a difference profile according to the invention, a threshold value which corresponds to approximately one and a half times, preferably approximately two times, more preferably approximately three times the signal to noise ratio can very suitably be used in the normalized spectrum. Here, noise in the NMR spectrum is understood to mean the signals coming from aspecific measurement events, such as for instance machine noise, environmental fluctuations, and/or contaminations in the chemicals.

It is also possible to use the value of the average signal intensity of 60-99%, preferably 70-95%, more preferably 80-90% of all spectral line positions showing a change in intensity between healthy individuals and ill individuals as a threshold value for obtaining difference profile according to the invention.

The choice for the level of the threshold value will also inter alia depend on the individual properties of the mammal for which the difference profile is determined. Such properties comprise sex, age, stage of life (fertile/infertile), diet, possible medication, genetic background, and, in humans, tobacco and/or alcohol consumption. The use of homogeneous groups of people is preferred in the methods according to the invention described hereinbelow, with a homogeneous group being defined as a group of individuals with as many comparable properties as possible, the only difference being the presence or absence of the disease.

Preferably, a normalized spectrum of metabolites in a body fluid of a mammal comprises a set of data coming from a homogeneous group of individuals. That means that a difference profile according to the invention for detection of a disease in a male individual comprises NMR spectral line positions with corresponding signal intensities of preferably exclusively male individuals. A difference profile for a disease can therefore be different depending on the properties of the individuals from which it has been obtained.

Preferably, a normalized spectrum of metabolites in a body fluid of a mammal represents a set of data coming from at least two, more preferably at least three, still more preferably at least four, and even more preferably at least five individuals.

A difference profile can very suitably comprise 3 to 1,000 spectral line positions corresponding to possibly original spectral lines. Preferably, a difference profile according to the invention comprises 10 to 500, more preferably 15 to 100, and still more preferably 20 to 70 spectral line positions. Very good results have been obtained with a difference profile comprising 30 to 50 spectral line positions.

The number of spectral line positions from which the difference profile is built up is chiefly determined by the definition of the threshold value mentioned. This threshold value, in which the value for the pitch of the noise in the normalized spectra can have been taken into account, indicates from which value differences in the height of a spectral line between individuals in which a disease has been diagnosed and healthy individuals are “significant”. A difference in height can be either positive (increase of intensity) or negative (decrease of intensity).

As said, the detection of a disease by means of a difference profile according to the invention is preferably used in individuals with properties which are corresponding or similar to those of the individuals from which the difference profile has been obtained, but this is by no means necessary.

The present invention also relates to a method for manufacturing a difference profile for the detection of a disease in a mammal.

A difference profile according to the invention can very suitably be manufactured by means of a method comprising the step of providing a first set of positions and corresponding intensities of spectral lines in an NMR spectrum which has been recorded from metabolites in a body fluid of healthy individuals of a mammal.

As a body fluid which can be used in a method according to the invention, in principle, any body fluid can be used. Preferably, a body fluid is used which can be obtained in a non-invasive manner. It is most preferred that the body fluid be urine.

Although, in embodiments of the present invention, in principle, different measurement methods for measuring metabolites in a body fluid can be used, preferably proton nuclear magnetic resonance spectroscopy is used. An NMR instrument with a frequency of at least approximately 200 MHz is, in principle, suitable, but there is a preference for use of instruments with a higher frequency, such as at least approximately 300 MHz, more preferably at least approximately 400-600 MHz.

For carrying out NMR spectroscopic analysis, samples of a body fluid can very suitably be lyophilized and the lyoplilisate can then be reconstructed in a suitable buffer, for instance a sodium phosphate buffer, which is prepared on the basis of D₂O. A suitable acid content for such a buffer is in the range of pH 4-10, preferably of pH 4-8, and more preferably, such a buffer has a pH of approximately 6. Preferably, different samples which will be mutually compared are reconstructed in buffers of equal pH. The reconstitution of the lyophilized components of a sample of a body fluid in a buffer of equal pH serves to minimize spectral differences caused by differences in pH between different samples. To the reconstructed sample, further, an internal standard, such as for instance TMSP (sodium trimethylsilyl-[2,2,3,3,-²H₄]-1-propionate) or tetramethylsilane can be added. Then, an NMR spectrum can be recorded from these samples, the NMR instrument being set for ¹H NMR analysis. Preferably, an NMR spectrum of a sample is recorded in triplicate. In general, default settings as recommended by the manufacturer can be used for this purpose. The measurement results are shown in chemical shift in relation to the internal standard and are expressed in “ppm” (parts per million).

In the present invention, a spectral line position is expressed in “ppm”, while the signal intensity is expressed in “regression” (see also FIG. 2), as is conventional in the field.

To the recorded spectra, optionally, a manual baseline correction is applied and the spectra are then processed into so-called line listings by means of standard NMR procedures. For this purpose, all lines in the spectra above the noise are collected and converted into a data file which is suitable for multivariate data analysis.

Preferably, several healthy individuals of the respective mammal are measured so that glitches can be arithmetically taken into account. Such an arithmetic account of glitches can very suitably take place in combination with the process of normalization of the measurement data. For determining a normalized spectrum of metabolites in a body fluid of a healthy mammal, in principle, one single healthy individual can be measured, but preferably, spectra coming from a group of healthy individuals are used, more preferably a homogeneous group.

Normalization of several recorded NMR spectra contributes to the reliability of a set of values obtained from a plurality of individuals. Further, normalization allows the comparison of a separately recorded spectrum with a set of previously recorded spectra.

A method for manufacturing a difference profile also comprises the step of providing a second set of positions and corresponding signal intensities of spectral lines in an NMR spectrum which has been recorded in a corresponding manner from metabolites in a corresponding body fluid of individuals of that same mammal in which the specific disease has been diagnosed.

Preferably, here as well, several individuals of a homogeneous group of the respective mammal in which the respective disease has been diagnosed are measured so that glitches can be arithmetically taken into account. To the recorded spectra, optionally, a manual baseline correction is applied and the spectra are then processed into so-called line listings by means of standard NMR procedures. For this purpose, all lines in the spectra above the noise are collected and converted into a data file which is suitable for multivariate data analysis. The recorded NMR spectra are preferably normalized in the above-described manner.

Finally, a method for manufacturing a difference profile comprises the step of comparing the normalized spectral line intensities corresponding to corresponding spectral line positions in the first and second set of positions of spectral lines in an NMR spectrum, and detecting the differences between them for obtaining a difference profile according to the invention.

Multivariate data analysis or pattern recognition can very suitably be used to visualize differences related to disease and treatment in these spectra. The arithmetic method based on the Partial-Linear-Fit algorithm as described in WO 02/13228 is particularly preferred. This algorithm enables adjustment of small variations in the position of the spectral line in NMR spectra without loss of resolution.

The above-described Partial-Linear-Fit algorithm comprises a principal component discriminant analysis (PCDA) part. Here, the number of variables is first reduced by means of principal component analysis (PCA). The projections, so-called scores, of samples on the first principal components (PCs) are used as a starting point for linear discriminant analysis. The scores of the samples are plotted in a score plot, where similar samples tend to cluster and dissimilar samples will be spaced a larger distance from each other (see FIG. 1). The relation of discriminant axes to the original variables (NMR signals) is visualized in a loading plot. Here, the position of the original variables is shown so that the length of the variable vector parallel to a discriminant is proportional to the loading of that variable to that axis.

Another possibility to visualize the data is by means of factor spectra (see inter alia FIG. 2), which correlate to the positions of clusters in score plots (e.g. the osteoarthritis cluster in FIG. 1) by graphical rotation of loading vectors. These factor spectra, or metabolic fingerprints, made in the direction of maximum separation of one category in relation to another category, provide insight in the types of metabolites responsible for separation between the categories.

Therefore, a difference profile according to the present invention can very suitably be shown as a factor spectrum, an example of which is shown in FIG. 2, or as a table with spectral line positions, an example of which is shown in Table 1 below.

TABLE 1
Characteristic increasing and decreasing NMR
spectral line positions due to osteoarthritis

| NMR spectral line positions with | NMR spectral line positions with |
|---|---|
| increasing values due to | decreasing values due to |
| osteoarthritis in ppm ±0.05 | osteoarthritis in ppm ±0.05 |
| 1.35 | 0.93 |
| 1.65 | 1.20 |
| 2.00 | 1.33 |
| 2.17 | 1.45 |
| 2.23 | 1.48 |
| 2.42 | 1.90 |
| 2.45 | 2.75 |
| 2.48 | 3.20 |
| 2.50 | 4.13 |
| 2.58 | 6.63 |
| 2.80 | 7.30 |
| 2.88 | 8.32 |
| 2.90 | |
| 3.02 | |
| 3.08 | |
| 3.10 | |
| 3.30 | |
| 3.50 | |
| 3.65 | |
| 4.03 | |
| 4.33 | |
| 5.97 | |
| 6.42 | |
| 6.45 | |
| 6.78 | |

Since, in the present invention, the analytical methodology of proton nuclear magnetic resonance spectroscopy is used for obtaining numeric data concerning metabolites, the values obtained depend on the settings of the instrument and the conditions under which the measurement is carried out. Also, the absolute values depend on the reference (e.g. the internal standard) used in the measurement. A difference profile, examples of which are shown in Table 1, thus comprises values which can differ between different measurement moments and between different measurement conditions. For this reason, the values as shown in Table 1 are not absolute values. The meaning of the individual values of both the spectral line positions and the possible spectral line intensities in the difference profile for osteoarthritis thus substantially resides in their ratio and position in relation to each other and therefore in the pattern of these values.

Due to deviant measurement conditions as indicated hereinabove, the ppm value of a spectral line defined in Table 1 can be located at a point with a ppm value of ±0.05 ppm as shown in Table 1.

The present invention further relates to a method for the detection of a disease in a mammal, comprising the steps of providing an NMR spectrum of metabolites in a body fluid of an individual of this mammal in which a particular disease is suspected and comparing this NMR spectrum with one or more difference profiles for particular diseases determined according to the invention for a corresponding body fluid in a corresponding mammal. Such a comparison step can be carried out visually, but also arithmetically.

Diseases which can be detected by means of the present invention are, for instance, immunological diseases and (chronic) inflammatory diseases, degenerative processes (and of course also regenerative recovery processes), cancer, and/or systemic diseases such as systemic lupus erythematosus (SLE), rheumatoid arthritis (RA) or systemic sclerosis. A non-exhaustive list of diseases which can be diagnosed and prognosticated using the present invention is shown in Table 2.

TABLE 2
Diseases which can be diagnosed and prognosticated
by means of the present invention.

Immunological diseases, Systemic diseases and (chronic)
Inflammatory diseases
Active chronic hepatitis
Allergies
Angioneurotic edema
Anti-phospholipid syndrome
Anti-phospholipid syndrome
Temporal arteritis
Ataxia Telangiectasia
Autoimmune gastritis
Autoimmune hemolytic anemia
Autoimmune hepatitis
C1-esterase deficiency
Chediak Higashi Syndrome
Chronic granulomatous disease (CGD)
Chronic fatigue syndrome (CFS, ME)
Chronic mucocutaneous candidiasis
Chronic sinusitis
Ulcerative colitis
Complement deficiency
CREST syndrome
Cryoglobulinemia
CVID (Common variable immunodeficiency)
Dermatomyositis
Diabetes mellitus type I
Discoid lupus erythematosus
Febris e.c.i.(fever of unknown origin)
Sex-linked agammaglobulinemia (XLA)
Homocystinuria
Hypereosinophilic syndrome
Hyper IgM
Hyper-IgD syndrome
Hyper-IgE syndrome
Hypersensitivity vasculitis
Hypogammaglobulinemia
Idiopathic thrombocytopenic purpura
IgA- deficiency
IgG subclass deficiency
Immunodeficiency with thymoma
Infections of unknown origin
Interstitial cystitis
Keratoconjunctivitis sicca
Leukopenia of unknown origin
Leukocyte adhesion deficiency
Disseminated lupus erythematosus
Lupus-like syndrome
Microscopic polyangiitis (MPA)
Mixed connective tissue disease (MCTD)
Multiple sclerosis
Myasthenia gravis
Myeloperoxidase (MPO) deficiency
Pemphigus
Pernicious anemia
Polyangiitis overlap syndrome
Polyarteritis nodosa (PAN)
Polymyalgia rheumatica
Polymyositis
Primary myxedema
Primary biliary cirrhosis
Primary neutropenia
Progressive systemic sclerosis
Relapsing polychondritis
Rheumatoid arthritis
Giant cell arteritis
Sarcoidosis (Besnier-Boeck disease)
SCID (Severe Combined Immunodeficiency)
Scleroderma polymyositis
Scleroderma
Subacute cutaneous lupus erythematosus
Ehlers-Danlos Syndrome
Goodpasture Syndrome
Marfan's Syndrome
Sjogren's Syndrome
Stickler Syndrome
Systemic lupus erythematosus (SLE)
Uveitis
Vasculitis
Wiskott-Aldrich syndrome
Wiskott-Aldrich syndrome
X-linked agammaglobulinemia
X-linked lymphoproliferative syndrome
Addison's disease
Behcet's disease
Churg-Strauss syndrome
Crohn's disease
Ophthalmic Graves' disease
Hashimoto's disease
Reiter's disease
Takayasu arteritis
Tietze's disease
Wegener's disease
Cancer
Pancreas cancer
Cervical cancer
Uterine cancer
Bladder cancer
Bone marrow cancer
Breast cancer
Intestinal cancer
Colon cancer
Skin cancer
Throat cancer
Leukemia
Liver cancer
Lung cancer
Stomach cancer
Kidney cancer
Pancreas cancer
Prostate cancer
Thyroid cancer
Testicular cancer
Hodgkin's disease
Kahler's disease
Infectious diseases
Bovine brucellosis
Blue ear disease
Actinomycosis
Rheumatic fever
African horse sickness
African swine fever
AIDS
Equine encephalomyelitis
Anaplasma infection
Atrophic rhinitis
Bacillar hemoglobinemia
Contagious agalactia
Contagious bovine pleuropneumonia
Contagious epididymitis
Contagious lymphatic vessel inflammation
Contagious equine metritis
Contagious pleuropneumonia
Pig typhoid
Echinoccosis Hydatidosis
Purpura hemorrhagica
Bovine malignant catarrh
Shipping fever
Botulism
Bovine Spongiforme Encephalopathy (BSE)
Bovine virus diarrhea (BVD)
Bronchitis
Bronchopneumonia
Typhoid
Chlamydophila abortus
Cholera
Coccidiosis
Conjunctivitis
Coxsackie virus infection
Cryptosporidiosis
Cysticercosis
Cytomegalia
Intestinal infection
Dermatophilosis
Diphtheria
Dourine
Dysentery
Encephalitis
Enzootic bovine leukosis
Enterocolitis
Enterotoxemia
Erythema infectiosum
Typhoid fever
Gas gangrene
Gastritis
Giardiasis
Gingivitis
Influenza
Heartwater
Hepatitis
Herpes
Herpes zoster
HIV infection
Rabies
HPV infection
Infectious anemia
Neonatal septicemia of sucklings
Keratoconjunctivitis
Classical swine fever
Classical fowl plague
Infectious bovine rhinotracheitis
Legionella pneumonia
Leptospirosis
Liver fluke
Lasteriosis
Longadenomatosis
Pneumonia
Lyme disease
Malaria
Malta fever (brucellosis)
Mastitis
Meningitis
Meningoencephalitis
Metritis
Anthrax
Foot-and-mouth disease
Epidemic myalgia
Myositis
Myxomatosis
Nairobi sheep disease
Necrobacillosis
Nodular dermatosis
Equine influenza
Paradontitis
Parasitic diseases
Paratuberculosis infection
Paratyphoid fever
Peritonitis
Piroplasmosis
Pleuropneumonia
Pododermatitis
Smallpox
Poliomyelitis
Polyarthritis
Polyserositis
Pseudo foot-and-mouth disease
Pseudo membranous enteritis
Pseudo tuberculosis
Newcastle disease
Pulpy nephritis
Q-fever
Rhinitis
Rhinopneumonia
Rift Valley fever
Rinderpest
Fungous infections
Scrapie
Neonatal septicemia
Sexually transmitted diseases
Streptococci group A-infections
Streptococci group B-infections
Subacute bacterial endocarditis
Teschen disease
Tetanos
Tetanus
Theileriosis
Toxic shock syndrome
Toxoplasmosis
Trichinellosis
Trichinosis
Trichomonas infection
Trypanosomiasis
Tuberculosis
Tularemia
Vesiculitis
Viral hemorrhagic disease
Viral myocarditis
Spotted fever
Swine erysipelas
Erysipelas
Bornholm disease
Carré's disease
Creutzfeldt-Jakob disease
Glässer's disease
Mucocutaneous lymph node syndrome
Glandular fever
Tyzzer's disease
Weil's disease
Zoonoses
Maedi-Visna
Degenerative diseases
Amyotrophic lateral sclerosis (Charcot's disease)
Atherosclerosis
Chromosome 17-linked dementia
Corticobasal degeneration
Cystic Fibiosis/Mucoviscidosis
Diffuse Lewy body diseases
Discopathy
Frontotemporal dementia
Lewy body dementia
Osteoarthritis
Osteomyelitis
Osteoporosis
Primary progressive aphasia
Progressive supranuclear palsy (PSP)
Progressive muscular dystrophy (Duchenne muscular dystrophy)
Spondylarthrosis
Vascular dementia
Alzheimer's disease
Binswanger's disease
Creutzfeldt-Jakob disease
Gaucher's disease
Huntington's disease
Korsakoff's disease
Parkinson's disease
Pick's disease
Pompe's disease

It is possible, but not necessary, to normalize the NMR spectrum of metabolites in a body fluid of an individual of this mammal in which the disease is suspected prior to comparing it with a difference profile according to the invention by means of spectra of metabolites in a body fluid of healthy individuals of the respective mammal. If it appears from the comparison step that the characteristic difference profile is really comprised in the spectrum recorded from an individual in which the disease is suspected, the presence of the disease is thus determined.

It is also possible to plot the data of the spectrum recorded from an individual in which the disease is suspected in a score plot, such as for instance the score plot of FIG. 1, and to determine whether the data fall within the cluster of “ill” spectra. If these data of an individual in which the respective disease is suspected do not fall within the cluster designated “ill”, the respective disease is not present in the respective individual, at least not at the disease stage as it occurred in the “ill” reference group. In the present invention, such a diagnostic method step is understood to be comprised in the step for comparing an NMR spectrum with a difference profile.

Metabolites are conversion products of organic compounds which are found in the body in different forms and numbers. For instance, in a healthy body, the ratio and the occurrence of metabolites in a body fluid, such as urine or blood, are totally different than in an unhealthy body. In urine, such metabolites are considered waste products.

In the present context, biomarkers are understood to mean one or more organic compounds or their metabolites, or specific patterns or specific amounts of several organic compounds or their metabolites, which can be found in the body of a mammal and which are the result of a subclinical or clinical event in that body.

By measuring biomarkers in a body fluid of a mammal, it is possible to quickly distinguish an unhealthy or ill condition from a healthy condition. The present invention provides a method for the identification of biomarkers.

A biomarker according to the invention can be one single substance or metabolite, but also a specific combination of substances or metabolites. In the latter case, it can also be considered a set of biomarkers. Preferably, according to the present invention, a biomarker is a specific combination of metabolites which, as a result of the disease, can be found in a specific pattern of concentrations or amounts in a body fluid, preferably urine, and which can be derived from a difference profile. In the present invention, a biomarker is also understood to mean moieties of organic compounds or of metabolites.

The present invention provides a method for the identification of a biomarker for a particular disease, comprising manufacturing a difference profile for that specific disease according to the invention and identifying one or more metabolites characterized by one or more defined spectral lines in this difference profile.

The identification of a metabolite which is characterized by one or more defined spectral lines in a difference profile can, for instance, be done by the coupling of a mass spectrometer to an NMR instrument and the subsequent analysis of the metabolite corresponding to one or more defined spectral lines by means of mass spectrometry (MS). A skilled person is familiar with mass spectrometry for the identification of organic compounds and metabolites. However, determining the identity of a metabolite corresponding to one or more defined spectral lines can also be done by recording the NMR spectrum from known metabolites and comparing it to the NMR spectral lines in a difference profile according to the invention.

For identifying the metabolites corresponding to the different spectral line positions, for instance, also the handbooks such as “Sadtler Standard Spectra series on NMR spectra” and “Aldrich Library of NMR Spectra” or other database files for ¹H NMR spectra can be consulted.

The ¹H chemical shifts of particular characteristic metabolites are, for instance (values ±0.05 ppm): N-acetylaspartate (CH3) appears as a singlet at 2.05 ppm (designation CH3), and as a multiplet at 2.91 and 1.95 ppm (designation CH2); inositol appears as a doublet at 3.25 ppm (designation H1/H3) and as a triplet at 4.10 ppm (designation H2); choline appears as a multiplet at 3.19 ppm (designation NCH2) and as a multiplet at 3.94 ppm (designation OCH2); neopterin appears as a multiplet at 4.34 and 4.44 ppm and (designation CH2), as a multiplet at 4.60 and 4.70 ppm (designation CH), and as a singlet at 5.20 ppm (designation OCH2), and taurine appears as a triplet at 3.26 ppm (designation CH2SO3) and 3.31 ppm (designation NCH2). Such metabolites can very suitably be used as biomarkers according to the present invention for detecting a disease in a patient, where increases in the concentration of the biomarkers indicate, for instance, the (increased) degradation or conversion of the base material from which these metabolites originate.

It could be determined that a difference profile according to the invention, a representative example of which is shown in Table 1 and FIG. 2, contains a different pattern of spectral lines with a positive regression. (i.e. spectral lines whose height has increased) and spectral lines with a negative regression (i.e. spectral lines whose height has decreased) for individual diseases, which spectral lines are characteristic of specific metabolites.

For instance, it could be determined that the spectral lines which show an increase in intensity in FIG. 2 (factor spectrum for osteoarthritis) are specific for lactic acid, malic acid, mercapturic acid and/or acetyl cysteine and monophosphates.

It is assumed that these metabolites are secreted in the urine as a result of the disease, and the accompanying complex physiological degradation and inflammatory symptoms, and that thus, the secretion of these metabolites in the urine is specific for the presence of the disease.

Metabolites that are found in increased amounts in a body fluid, e.g. the urine, of patients which are examined for the presence of a disease can very suitably be used as a biomarker. Metabolites which decrease in amount in ill individuals in relation to healthy individuals can less well be applied as a biomarker due to the danger of false negative results in particular detection methods. Metabolites with a positive regression in a difference profile according to the invention are therefore preferably used as a biomarker in a system for the rapid and early detection of a disease.

In many cases, it will not be possible to conclude from the difference profile whether the metabolites are secreted in the urine in free condition or in a derived form, for instance conjugated or bound in another manner. However, a skilled person will understand that the metabolites described can be used as a biomarker in any condition in which they may be found in the body fluid.

The invention also relates to biomarkers for diagnosis and prognosis of osteoarthritis, available by using a method according to the invention. In particular, the present invention provides a biomarker for osteoarthritis characterized in that this biomarker is formed by a metabolite containing at least one compound, which metabolite is chosen from the group consisting of lactic acid, malic acid, mercapturic acid, acetyl cysteine, monophosphate compounds and their functional analogs.

The present invention further relates to a method for the detection (i.e. the diagnosis and/or prognosis) of a disease in a mammal, comprising measuring a biomarker according to the invention in a body fluid, preferably urine. Such a measurement is preferably non-invasive, and preferably comprises the detection, in a body fluid of an individual of a mammal in which a disease is suspected, of a quantitative change in the occurrence of a biomarker in relation to a normal value for that biomarker which is found in a body fluid of healthy individuals and which quantitative change corresponds to the regression of that biomarker in the difference profile for the respective disease.

A measurement of a biomarker can also comprise the detection of a pattern of concentrations or amounts of metabolites in a body fluid of an individual of a mammal in which a disease is suspected in the case that the biomarker is a pattern of several metabolite concentrations. If such a pattern of concentrations or amounts of metabolites, which pattern is measured in the form of a biomarker measurement in an individual of a mammal, corresponds to the difference profile of the respective disease for which the biomarker has been determined, the disease is present in that individual. In that case, a qualitative biomarker measurement is involved.

So, a method for detection of a disease in a mammal according to the invention comprises the quantitative or qualitative detection of a biomarker according to the invention in a body fluid of that individual.

A measurement of a biomarker for detection of a disease in a mammal according to the invention is preferably carried out for urine.

A measurement of a biomarker in a body fluid of an individual of a mammal for the detection of a disease according to the invention will always comprise the step of comparing the measurement value found to a reference, which reference can comprise a characteristic value for healthy individuals and/or a characteristic value for individuals in which the respective disease has been diagnosed.

A diagnose can be made on the basis of the results of the measurement of a biomarker according to the invention. For instance, a normal level of metabolites or a normal pattern of metabolites will provide the diagnosis “healthy”. Conversely, an undesired metabolite pattern or an undesired metabolite level will provide the diagnosis “ill”, where, depending on the specificity and nature of the disease-specific marker used, the name of the disease is known.

By means of the present invention, it is therefore possible to detect a disease in a mammal by observing specific biochemical changes in the body fluid of an individual of a mammal, which changes are preferably detected by measurement of a biomarker according to the invention.

A biomarker according to the invention can be measured in a body fluid in different manners. For instance, NMR and/or Mass Spectrometry (MS) can be applied to a sample of a body fluid. But other analytical methods can also be used for this purpose, such as ELISA or a related methodology.

An even simpler and more rapid diagnosis can be made by using microsystem technologies, for instance by using a “microfluidics” instrument or a microelectromechanic system (MEMS) in combination with, for instance, specific fluorescent enzymes or other manners of detection by means of which the biomarkers found in a body fluid can be quantitatively and/or qualitatively measured. A skilled person will be able, without many problems, to acquaint himself with the state of the art in the area of the rapid detection of biomarkers and/or metabolites and is able to formulate methods for measuring biomarkers according to the present invention in a body fluid of a mammal for the diagnosis or prognosis of a disease. (See for instance M. Madou, Fundamentals of Microfabrication: The Science of Miniaturization, 2^{nd} Ed, CRC Press 1997; N. Nam-Trung & S. Wereley, 2002, Fundamentals and Applications of Microfluidics, Artech House Publishers; J. W. Gardner et al., 2001, Microsensors, MEMS and Smart Devices, Wiley, Chichester).

The present invention also relates to an apparatus for using a method for the detection of a disease in a mammal by measuring a biomarker according to the invention. Such an apparatus preferably comprises a solid carrier with immobilized binding partners for this biomarker thereon. The nature of such binding partners depends on the biomarker which will be measured, but can, for instance, comprise an antibody or a peptide as a specific binding partner which is able to specifically bind the biomarker. An apparatus according to the invention further preferably comprises a system for quantitative detection of binding between the biomarker and the immobilized binding partners. Such a system can comprise either direct detection (for instance by applying fluorescent labels on the biomarker) or indirect detection (for instance by applying a secondary binding partner to the biomarker, which secondary binding partner comprises a detectable label). A skilled person is assumed to be familiar with systems and methods for bringing about a bond between an immobilized binding partner and a biomarker according to the invention and the systems for detecting the bond between them.

By means of the present systems and methods, a disease can be diagnosed in a qualitative manner. For this purpose, for instance, a database is compiled of NMR spectra recorded from substantially all metabolites in a body fluid, preferably urine, of one or more individuals with a defined disease, such as one or more NMR spectra of, for instance, osteoarthritis patients and/or one or NMR spectra of patients suffering from a disease according to Table 2, for instance multiple sclerosis patients. The known NMR spectra of such a database can be compared to an NMR spectrum recorded from a patient in which a particular disease, for instance osteoarthritis, is suspected. If such a database only comprises one or more NMR spectra of patients suffering from osteoarthritis, only qualitative detection of osteoarthritis is possible by means of such a database. If, however, the database contains NMR spectra of a large number of different and defined diseases, in a patient in which a disease is suspected, the qualitative detection of a large number of diseases will be possible. It will be possible to carry out this detection by comparison of an NMR spectrum of this patient suffering from an unknown disease with the NMR spectra in the database. If such a comparison step yields a match with NMR spectra in the database for a specific disease, the disease is thus demonstrated in this respective patient. A database base comprises such NMR spectra preferably in normalized form.

In addition to the fact that a database according to the invention can comprise one ore more optionally normalized NMR spectra for one or more defined diseases, such a database can instead comprise one or more difference profiles according to the invention, formulated for one or more defined diseases.

The use of difference profiles in a database according to the invention has the advantage that the database will comprise considerably fewer data than in the case in which complete, optionally normalized, NMR spectra are stored in it.

By formulating difference profiles for one or more diseases at different stages of progression and including them in a database, a quantitative series of difference profiles for quantitative analysis of a disease can be obtained. By carrying out a comparative analysis of an NMR spectrum of an individual in which a particular disease is suspected, or of which the severity of the disease is to be determined, this quantitative series of difference profiles can be used to quantitatively express the presence of a disease. Further, the progression of the disease can be quantitatively followed in this manner.

Therefore, the present invention also relates to database comprising one or more disease-specific difference profiles according to the invention, optionally with annotation of the stage of the disease.

Preferably, such a database comprises difference profiles for diseases which are difficult to diagnose or difficult to prognosticate. Very suitably, a database according to the invention comprises difference profiles for different types of cancer, leukemia, Parkinson's disease, Hodgkin's disease, Crohn's disease, Alzheimer's disease, AIDS, diabetes, tuberculosis, multiple sclerosis, amyotrophic lateral sclerosis, cerebrospinal meningitis, poliomyelitis, progressive muscular dystrophy, encephalitis, tetanus, viral hepatitis, malaria, spotted fever, typhoid fever, paratyphoid fever, diphtheria, cholera, anthrax, osteoarthritis, osteoporosis, allergies and/or mucoviscidosis.

It is also possible to use a biomarker according to the invention for quantitative analysis of a disease if the quantity in which this biomarker is found in a body fluid of ill individuals can be correlated to different stages of progression of the respective disease.

By using the present invention in combination with metabolic and physiological measurements, it is now, for instance, possible to diagnose and quantify osteoarthritis at an early stage. This analysis of osteoarthritis, the knowledge of the pathogenesis and the efficiency of therapies can greatly improve through use of the present invention. By using a biomarker according to the invention, such as a biomarker chosen from the group consisting of lactate, malate, β-alanine, hypoxanthine, 3,4-dihydroxy mandelate, 3-hydroxy cinnamic acid, alanine, aspargine or N-acetyl aspartate, alone, or in combination, it is thus now possible to detect osteoarthritis at an early stage and to improve the treatment of patients.

In principle, the invention can be applied to animals, including fishes, birds, and is preferably applied to mammals in general and to equines, bovines, porcines, ovines, myomorpha, canines, rodentia, simians and primates in particular. Preferably, the invention is applied to guinea pigs, dogs or humans.

The invention will be illustrated hereinbelow on the basis of an example.

### EXAMPLE 1

Sample Preprocessing

Prior to NMR spectroscopic analysis, 1 ml urine samples were lyophilized and reconstructed in 1 ml of sodium phosphate buffer (pH 6.0, based on D₂O) with 1 mM of sodium trimethylsilyl-[2,2,3,3,2H4]-1-propionate (TMSP) as an internal standard.

NMR Measurements

NMR spectra were recorded in triplicate in a fully automated manner on a Varian UNITY 400 MHz spectrometer provided with a proton NMR set-up and at a working temperature of 293 K. Free induction decays (FIDs) were collected as 64K data points with a spectral band width of 8,000 Hz; 45-degree pulses were used with a measurement time of 4.10 sec. and a relaxation delay of 2 sec. The spectra were determined by accumulation of 128 FIDs. The signal of the residual water was removed by a presaturation technique in which the water peak was irradiated with a constant frequency for 2 sec. prior to the measurement pulse.

The spectra were processed using the standard Varian software. An exponential window function with a line broadening of 0.5 Hz and a manual baseline correction was applied to all spectra.

After reference to the internal NMR standard (TMSP δ=0.0), line listings were compiled by means of the standard Varian NMR software. To obtain these line listings, all lines in the spectra with a signal intensity above the noise were collected and converted to a data file which was suitable for use of multivariate data analysis.

Determination of Metabolic Fingerprint or Difference Profile of Osteoarthritis Metabolites.

By means of a 400 MHz NMR spectrometer, urine samples were tested of healthy individuals and of individuals in which osteoarthritis had been diagnosed. The spectra were processed and line listings were compiled by means of standard Varian software after reference to the internal standard. The NMR data reduction file was imported into Winlin VI. 10. Small variations of comparable signals in different NMR spectra were adjusted by using the Partial-Linear-Fit algorithm as described in WO 02/13228 and the lines were fitted without loss in resolution. The scale of the data was automatically adjusted and “normalized” to unit intensity. The endogenous and exogenous metabolites were eliminated from the NMR spectra, which led to the reduction of the data to specific and “significant” osteoarthritis-related changes. For this purpose, a threshold value was used by means of which 80-90% of the spectral line positions were eliminated.

A score plot (FIG. 1) of the NMR spectra was made by means of multivariate data analysis as described hereinabove. From the score plot, a metabolic fingerprint or difference profile was obtained by selecting rising and falling NMR signals with relatively high frequency of occurrence in urine of osteoarthritis patients. From these, a choice was made of approximately 35 NMR signals with a relevant contribution to osteoarthritis (regression >0.5). These NMR signals are shown in Table 1 and FIG. 2.

1. A difference profile for the detection of a disease in a mammal, comprising a plurality of spectral line positions and optionally corresponding signal intensities of NMR spectral lines, which express the normalized difference between one or more NMR spectra of metabolites in a body fluid of one or more healthy individuals of said mammal, and one or more corresponding NMR spectra of metabolites in a corresponding body fluid of one or more individuals of said mammal in which said disease has been diagnosed.

2. A difference profile according to claim 1, wherein said mammal has been chosen from the group consisting of primates, dogs and rodents.

3. A difference profile according to claim 1, wherein said body fluid is urine.

4. A difference profile according to claim 1, wherein said disease is selected from the group consisting of an immunological disease, a (chronic) inflammatory disease, a degenerative disease, cancer, an infectious disease, a systemic disease.

5. A difference profile according to claim 1, wherein said disease is osteoarthritis.

6. A difference profile according to claim 5, comprising the spectral lines and values corresponding thereto according to Table 1.

7. A database comprising one or more difference profiles according to claim 1.

8. A database according to claim 7, wherein said mammal is a human.

9. A method for the detection of a disease in a mammal, comprising the steps of providing an NMR spectrum of metabolites in a body fluid of an individual of said mammal in which said disease is suspected and comparing said NMR spectrum with a difference profile from a database according to claim 7, which difference profile has been determined for a corresponding body fluid from a corresponding mammal.

10. A method according to claim 9, wherein said mammal has been chosen from the group consisting of primates, dogs and rodents.

11. A method according to claim 9, wherein said body fluid is urine.

12. A method according to claim 9, wherein said disease is osteoarthritis.

13. A method for manufacturing a difference profile for the detection of a disease in a mammal, comprising the steps of: a) providing a first normalized set of positions and corresponding signal intensities of spectral lines of one or more NMR spectra recorded from metabolites in a body fluid of one or more healthy individuals of said mammal; b) providing a second normalized set of positions and corresponding signal intensities of spectral lines of one or more NMR spectra recorded from metabolites in a corresponding body fluid of one or more individuals of said mammal in which said disease has been diagnosed; and c) detecting the spectral lines whose signal intensities differ between said first and second set, for obtaining said difference profile.

14. (canceled)

15. A method according to claim 13, wherein said disease is osteoarthritis.

16. A method for identifying a biomarker for a disease, comprising manufacturing a difference profile according to claim 1 and identifying one or more metabolites which are characterized by one or more defined spectral lines in said difference profile, which one or more metabolites, alone or in combination, characterize said biomarker.

17. A method according to claim 16, wherein said one or more metabolites are characterized by one or more defined spectral lines with a positive regression.

18. A method according to claim 16, wherein said disease is osteoarthritis.

19. A biomarker for the detection of a disease in a mammal, comprising one or more metabolites or parts thereof which are characterized by one or more defined spectral lines in a difference profile according to claim 1.

20. A biomarker for the detection of osteoarthritis, comprising one or more metabolites or parts thereof chosen from the group consisting of lactate, malate, β-alanine, hypoxanthine, 3,4-dihydroxy mandelate, 3-hydroxy cinnamic acid, alanine, aspargine and N-acetyl aspartate, and combinations thereof.

21. Use of a biomarker according to claim 19, for the detection of a disease in a mammal.

22. Use of a biomarker according to claim 20, for the detection of osteoarthritis in a mammal.

23. A method for detection of a disease in a mammal, comprising measuring a biomarker according to claim 19, in a body fluid of an individual of said mammal.

24. A method according to claim 23, wherein said body fluid is urine.

25. An apparatus for use of a method according to claim 23, comprising a solid carrier with one or more immobilized binding partners for said biomarker thereon.

26. An apparatus according to claim 25, further comprising a system for the quantitative detection of binding between said biomarker and said one or more immobilized binding partners.

27. A database comprising one or more difference profiles according to claim 4.

28. A database comprising one or more difference profiles according to claim 6.

29. A database according to claim 27, wherein said mammal is a human.

30. A database according to claim 28, wherein said mammal is a human.

31. A method for the detection of a disease in a mammal, comprising the steps of providing an NMR spectrum of metabolites in a body fluid of an individual of said mammal in which said disease is suspected and comparing said NMR spectrum with a difference profile from a database according to claim 8, which difference profile has been determined for a corresponding body fluid from a corresponding mammal.

32. A method according to claim 31, wherein said disease is osteoarthritis.

33. A method for identifying a biomarker for a disease, comprising manufacturing a difference profile according to claim 6 and identifying one or more metabolites which are characterized by one or more defined spectral lines in said difference profile, which one or more metabolites, alone or in combination, characterize said biomarker.

34. A biomarker for the detection of a disease in a mammal, comprising one or more metabolites or parts thereof which are characterized by one or more defined spectral lines in a difference profile according to claim 6.

35. A method for detection of a disease in a mammal, comprising measuring a biomarker according to claim 20 in a body fluid of an individual of said mammal.

36. An apparatus for use of a method according to claim 24, comprising a solid carrier with one or more immobilized binding partners for said biomarker thereon.

37. An apparatus according to claim 36, further comprising a system for the quantitative detection of binding between said biomarker and said one or more immobilized binding partners.
